(19) United States
(12) Patent Application Publication
(10) Pub. No.: US 2018/0211730 A1
(43) Pub. Date: Jul. 26, 2018

(54) HEALTH INFORMATION (DATA) MEDICAL COLLECTION, PROCESSING AND FEEDBACK CONTINUUM SYSTEMS AND METHODS

(71) Applicant: Arizona Board of Regents on Behalf of the University of Arizona, Tucson, AZ (US)

(72) Inventors: Marvin J. Slepian, Tucson, AZ (US); Fuad Rahman, Santa Clara, CA (US); Syed Hossainy, Hayward, CA (US)

(73) Assignee: Arizona Board of Regents on Behalf of the University of Arizona, Tuscon, AZ (US)

(21) Appl. No.: 15/746,765

(22) PCT Filed: Jul. 20, 2016
(86) PCT No.: PCT/US16/43177

§ 371 Date: Jan. 22, 2018

Related U.S. Application Data
(60) Provisional application No. 62/194,904, filed on Jul. 21, 2015.

(51) Int. Cl. G16H 50/70; G16H 10/60; G16H 80/00; A61B 5/00

(52) U.S. Cl. CPC G16H 50/70; A61B 5/0022; G16H 80/00; G16H 10/60

(57) ABSTRACT

A medical feedback continuum systems, method, and software product processes healthcare data of a plurality of patients collected from disparate sources to determine a patient medical model of one of the plurality of patients. A medical intensity status is generated from the patient medical model and displayed to a doctor during a consultation of the doctor with the one patient. Healthcare information is collected during the consultation and processed to determine an intended intervention prescribed by the doctor for the one patient. An outcome of the intervention is predicted based upon analytics of the patient medical model, and whether the predicted outcome of the intervention is favorable for the one patient is determined. If the predicted outcome of the intervention is not favorable, an intervention alert is generated and sent to the doctor during the consultation

### RELATED APPLICATIONS

This application claims priority to U.S. patent application Ser. No. 62/194,904, titled “Health Information (Data) Medical Collection, Processing and Feedback Continuum Systems and Methods”, filed Jul. 21, 2015, and incorporated herein in its entirety by reference.

### BACKGROUND

In modem healthcare computerization, the doctor is often restricted as to what information may be provided to, and is available within, healthcare computers and other digital information systems. Healthcare computers and the modern range of digital devices mostly provide data entry forms that require manual information entry in a certain format and within a certain space; for example the doctor uses a keyboard to type or dictate entries into a predefined textual data field. The amount of time the doctor is allotted for each patient is driven by many issues which have changed over the years including: increasing patient load, the rise in chronic disease conditions and economic circumstances, such as to enable insurance payments for each patient. Thus, the doctor typically has an increasing burden of patient number coupled with less time to spend on each patient and the amount of available data entry into the electronic medical records is reduced. In the past physicians would spend 30-60 min on a typical office encounter, now this is reduced to 10 min in the U.S. on average, and even less in several countries around the world. Similarly rounding in the hospital or clinic, or even in the home of field as a house call, is typically shorter today than in years past.

### SUMMARY

Beyond the above outlined progressive disconnect of increasing information and increasing patient burden versus less time available and more complex means of data entry—i.e. typing into structured forms, rather simply writing a “to the point essential note”—an opportunity exists to enter information relevant to a medical condition which is presently not being captured into the medical record to enhance documentation, aid diagnosis, provide population big data information and guide therapy. This data may be described as sensory, mobility and dynamic data—e.g. a clear odor, a tremulous movement, an audible respiratory noise, a visual grimace, an affect. This data maybe described and termed as “symptom and sign Metadata”—in the sense that this data may relate to a given symptom or sign—for example a patient may complain of reduced exercise capacity and upon walking in the office has a noticeably reduced gait, stride length and speed of walking—none of this typically enters the medical record.

The role of the health care encounter—whether it be the office, clinic, hospital, home or field, or any other location in which care is delivered—is critical in obtaining relevant information to steward, guide and otherwise direct the delivery of care and enhance the accuracy of care. Studies have repeatedly demonstrated over the years that despite the increased availability of complex, sophisticated diagnostic devices, instruments, lab tests, imaging systems and the like, that it is the history taking, the physician or health worker asking of questions—as to symptoms and signs, that is the most significant element in moving care forward. Studies have clearly demonstrated that more than 70% of diagnoses and advancement of care steps emanate from physician or health worker questioning of the patient. As such, about seventy percent of proper diagnoses for the patient are made by the doctor using non-computerized information, such as: what the patient says, how the patient looks and acts, how the patient behaves, how the patient sits, how they walk, how they smell, and other information gained by the doctor during one-on-one patient encounters and consultations. But this information is not known by the healthcare computers or other digital data systems. For example, where the same doctor consults with the patient on consecutive occasions, it is the doctor's memory and mental vision and reconstruction of previous consultations that helps the most in determining whether the patient's health is deteriorating, changing, or improving, and whether current treatment is effective. Where different doctors consult with the patient, information from previous consultations is often not available and the ‘newly on board’ physician has a less complete picture of the patient.

Today's healthcare is provided through many disparate services that collectively provide care to a patient. Each service collects and stores data for its future use, but shares only some data with other services. And, information that each service collects is often not usable by other services as that information is in a format not easily transferred and assimilated. Key factors in caring for the patient are therefore lost, resulting in additional procedures, hospital visits, and costs for both the patient and healthcare organizations.

In one embodiment, a health information medical collection, processing, and feedback continuum system, includes a knowledgebase, a plurality of transducers for continuously and/or periodically collecting healthcare data from disparate sources for a plurality of patients, an analytic engine capable of receiving and processing the healthcare data to continuously and/or periodically update the knowledgebase and to determine a patient medical model from the knowledgebase for one of the plurality of patients, and an interactive medical intensity status display for interactively displaying, based upon the patient medical model, one or more of a past medical status, a current medical status, and a predicted medical status of the one patient.

In another embodiment, a medical feedback continuum system includes a plurality of transducers for collecting medical information of a plurality of patients from disparate sources, a knowledgebase for storing the medical information, and an analyzer for processing the knowledgebase to determine a medical intensity status display indicative of health of one of the plurality of patients.

In another embodiment, a medical feedback continuum method receives, within a healthcare computer and from disparate sources, healthcare data for a plurality of patients. The healthcare data is processed to form normalized healthcare data which is stored within a knowledgebase. The knowledgebase is processed to determine a patient medical model for one of the plurality of patients based upon healthcare data of other of the plurality of patients having similar medical conditions to the one patient.

In another embodiment, a medical feedback continuum method processes healthcare data of a plurality of patients collected from disparate sources to determine a patient medical model of one of the plurality of patients. A medical intensity status is generated from the patient medical model and displayed to a doctor during a consultation of the doctor with the one patient. Healthcare information is collected during the consultation and processed to determine an intended intervention prescribed by the doctor for the one patient. An outcome of the intervention is predicted based upon analytics of the patient medical model, and whether the predicted outcome of the intervention is favorable for the one patient is determined. If the predicted outcome of the intervention is not favorable, an intervention alert is generated and sent to the doctor during the consultation.

### BRIEF DESCRIPTION OF THE FIGURES

FIG. 1 shows operation of a prior art medical information input system by a doctor during consultation with a patient.

FIG. 2 shows one exemplary medical feedback continuum system, in an embodiment.

FIG. 3 shows the transducer of FIG. 2 in further exemplary detail.

FIG. 4 is a schematic illustrating exemplary collection of healthcare information by the system of FIG. 2.

FIG. 5 shows the analytic engine of FIG. 2 including at least one data processing engine that processes received input data to create the knowledgebase, in an embodiment.

FIG. 6 is a schematic illustrating exemplary analysis of a natural language phrase to identify one concept of FIG. 5, in an embodiment.

FIG. 7 is a schematic illustrating exemplary inference, by the analyzer of FIG. 5, of one concept from two other concepts, in an embodiment.

FIG. 8 is a schematic showing one exemplary medical intensity status display of FIG. 2, generated from the patient medical model of FIG. 4, in an embodiment.

FIG. 9A is a schematic showing one exemplary medical intensity status display, generated from the patient medical model of FIG. 4, for a patient with heart disease, in an embodiment.

FIG. 9B shows one exemplary medical intensity status display resulting from selection of the displayed heart in the medical intensity status display of FIG. 9A.

FIG. 9C shows one exemplary medical intensity status display resulting from selection of a prediction with intervention button in the medical intensity status display of FIG. 9B.

FIG. 9D shows one exemplary medical intensity status display resulting from selection of a prediction without intervention button in the medical intensity status display of FIG. 9B (or from the medical intensity status display of FIG. 9C).

FIG. 9E is a schematic showing one exemplary medical intensity status display generated from the patient medical model of FIG. 4, for a patient with Asthma.

FIG. 9F shows one exemplary medical intensity status display resulting from selection of the displayed lungs in the medical intensity status display of FIG. 9E.

FIG. 9G shows one exemplary medical intensity status display resulting from selection of the prediction with intervention button in the medical intensity status display of FIG. 9F.

FIG. 9H shows one exemplary medical intensity status display resulting from selection of the prediction without intervention button in the medical intensity status display of FIG. 9F (or from the medical intensity status display of FIG. 9G).

FIGS. 9I through 9L show exemplary medical intensity status displays that graphically illustrate the difference between following interventions and not following interventions for various medical problems.

FIG. 9M shows one exemplary medical intensity status display resulting from selection of the avoid button in the medical intensity status display of FIG. 9E.

FIG. 10 shows one exemplary medical feedback continuum method, in an embodiment.

FIG. 11 shows exemplary sensors of the transducer of FIG. 2 used within a room, in an embodiment.

FIG. 12 is a flowchart illustrating one exemplary medical feedback continuum method, in an embodiment.

FIG. 13 shows one exemplary framework for implementing the analytic engine of FIGS. 2, 4, and 5 using an Apache Spark platform, in an embodiment.

### DETAILED DESCRIPTION OF THE EMBODIMENTS

To offset limitations of present-day healthcare computers, a doctor often creates handwritten notes for a patient's file. In the past, these notes typically were part of the medical record. Today, however, these handwritten notes are not available to others that provide care for the patient. Thus, patient care misses out on these impressions and comments and is thus not improved by use of such computers and electronic systems.

Medical feedback continuum systems and methods described hereinbelow provide feedback to both doctor and patient by collecting information—available but previously and/or presently not collected and/or otherwise lost—from caregivers and patients in a way that is faster and more convenient. As used herein, the term ‘continuum’ refers to the large quantity of healthcare information that is continually collected and processed to form a medical status of a patient. This continuum of information is collected from multiple disparate sources, converted into a standardized structured format, and stored within a database (sometimes denoted as knowledgebase hereinbelow). The database is then used to determine a complete (whole) health status of the patient and to predict medical events likely to occur for that patient based upon whether or not certain interventions are followed.

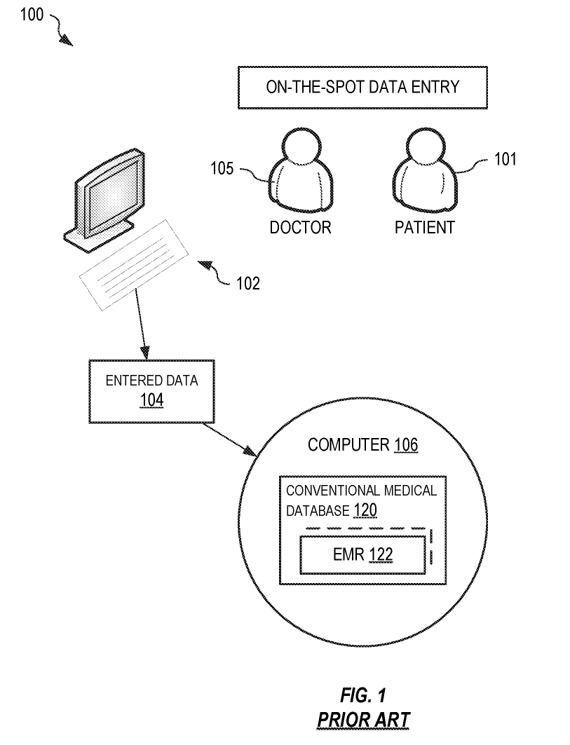

FIG. 1 shows operation of a prior art medical information input system 100 by a doctor 105 during a consultation with a patient 101. Doctor 105 is required to provide on-the-spot data entry to an input device (e.g., a computer terminal, a personal computer, or other similar device) such that an electronic medical record 122 for patient 101 is created within a conventional medical database 120 and stored within a computer 106. Specifically, doctor 105 enters information into a text field of input device 102 which sends the data to computer 106 for storing as EMR 122 within database 120. Such activity, however, is typically disruptive to interaction between doctor 105 and patient 101. Further, as noted above, input data 104 is not likely to contain all relevant information learned from patient 101 by doctor 105. Specifically, by looking at patient 101, doctor 105 learns important things about the patient's wellbeing. Where for example doctor 105 saw patient 105 on a previous visit, doctor 105 may compare his current impressions of that wellbeing against remembered impressions from the previous visit. However, where patient 101 sees a different doctor, that prior information is not available for comparison, and the doctor must rely upon EMR 122 made within the database 120.

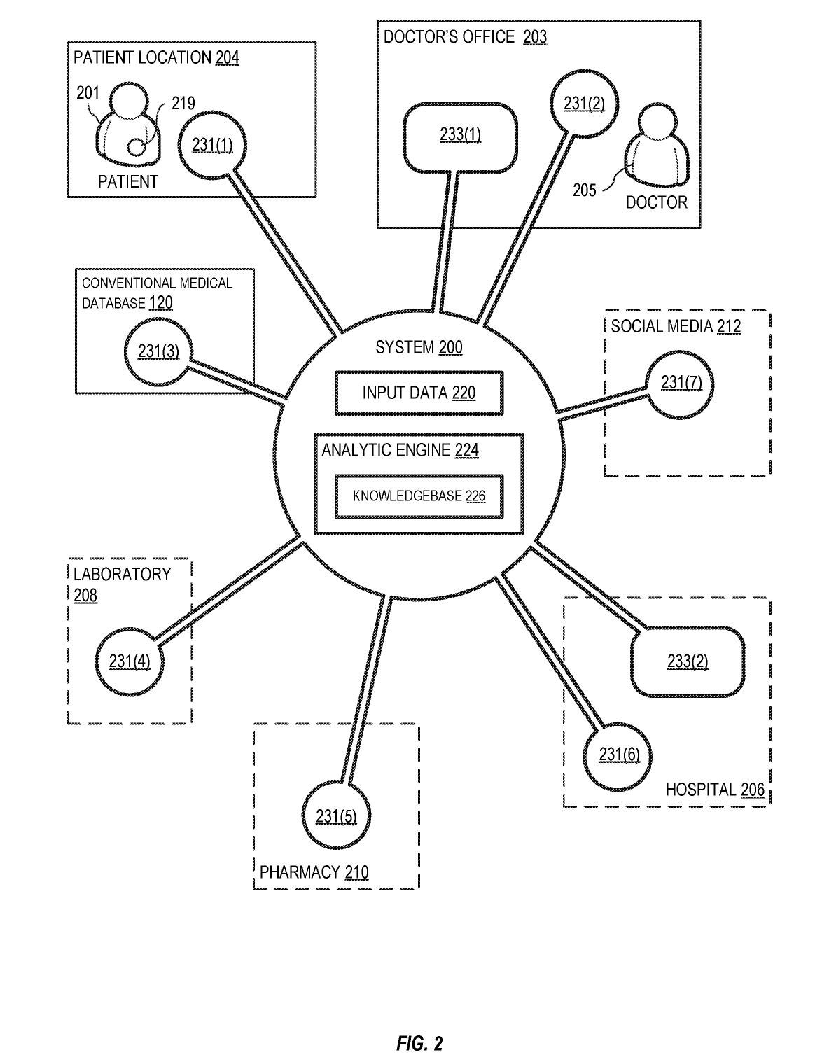

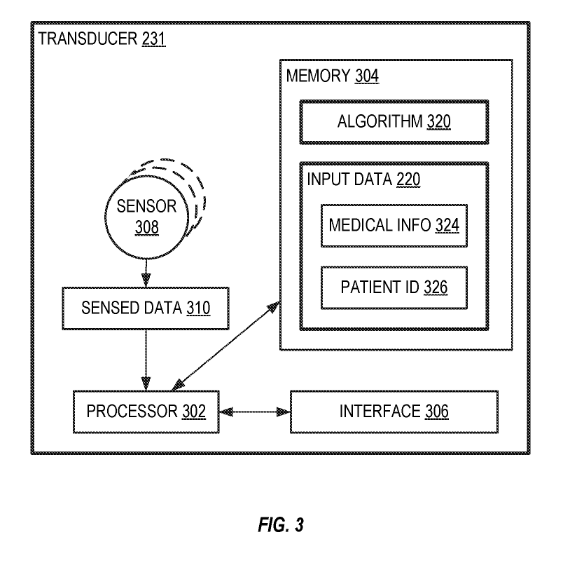

FIG. 2 shows one exemplary medical feedback continuum system 200. System 200 is for example a distributed computer that includes a plurality of transducers 231 that operate to collect input data 220 of a patient 201 for analysis by an analytic engine 224. A transducer 231(1) is located at a patient location 204 and operates to collect input data 220 at patient location 204. Patient location may represent any space where patient 201 may have a medical encounter where transducer 231 is present, including a consulting room, a home of the patient, a hospital, a care facility, a nursing facility, a rehabilitation facility, a convalescent care center, a skilled nursing facility, an assisted living facility, a long-term care facility, a hospice, and so on. For example, patient location 204 may represent a doctor's consulting room during a consultation between patient 201 and a doctor 205. In another example, patient location 204 represents a home of patient 201. Doctor 205 may or may not be proximate patient 201 during the medical encounter. FIG. 3 shows transducer 231 in further exemplary detail. FIGS. 2 and 3 are best viewed together with the following information.

Transducer 231 includes a processor 302, a memory 304, an interface 306, and one or more sensors 308. Sensors 308 may include one or more sensors selected from the group including: a sound sensor, a vibration sensor, an image sensor; an olfactory sensor, a motion sensor, a taste sensor, a temperature sensor, a humidity, hydration sensor, a compliance sensor, a stiffness sensor, and a pressure sensor, a microphone, a camera, a scanner, a touch sensor, a wearable sensor, an implanted sensor, and so on. Sensors 308 operate under control of processor 302 to collect sensed data 310, which is optionally processed by an algorithm 320, formed of machine readable instructions stored within memory 304 and executable by processor 302, to form medical information 324 within input data 220. Input data 220 may also include a patient ID 326 that is for example determined by interface 306. In one embodiment, interface 306 is a user interface for receiving patient ID 326 from doctor 205 or from an associated organization (e.g., hospital). In embodiments, data 220 includes information relevant to a medical condition which is presently not being captured into the medical record to enhance documentation, aid diagnosis, provide population big data information and guide therapy. This data may be sensory, mobility and/or dynamic data—e.g. a clear odor, a tremulous movement, an audible respiratory noise, a visual grimace, an affect—and may include asked data, evoked data, detected data, symptom data, sign data, lab data, imaging data, test data, as well as sensory data. In another embodiment, interface 306 is a wireless transceiver that interrogates an RFID tag associated with patient 201. For example, patient 201 may carry an ID card configured with the RFID tag that is encoded with patient ID 326. In another embodiment, patient 201 is recognized through recognition software associated with a sensor 308. In another embodiment, distributed system 200 automatically recognizes patient 201 based upon sensed biometrics of patient 201, such as through facial recognition, fingerprint recognition, iris recognition, and so on. In another embodiment, transducer 231 is a panel configured with sensors and couplers that may be permanently configured within a room (e.g., consulting room). In another embodiment, transducer 231 is implemented using a smart phone that has one or more communicatively coupled sensors (e.g., internal sensors of the smart phone and external sensors coupled therewith) that cooperate to collect medical information 324. In another embodiment, transducer 231 is a portable device that may be transported to patient location 204.

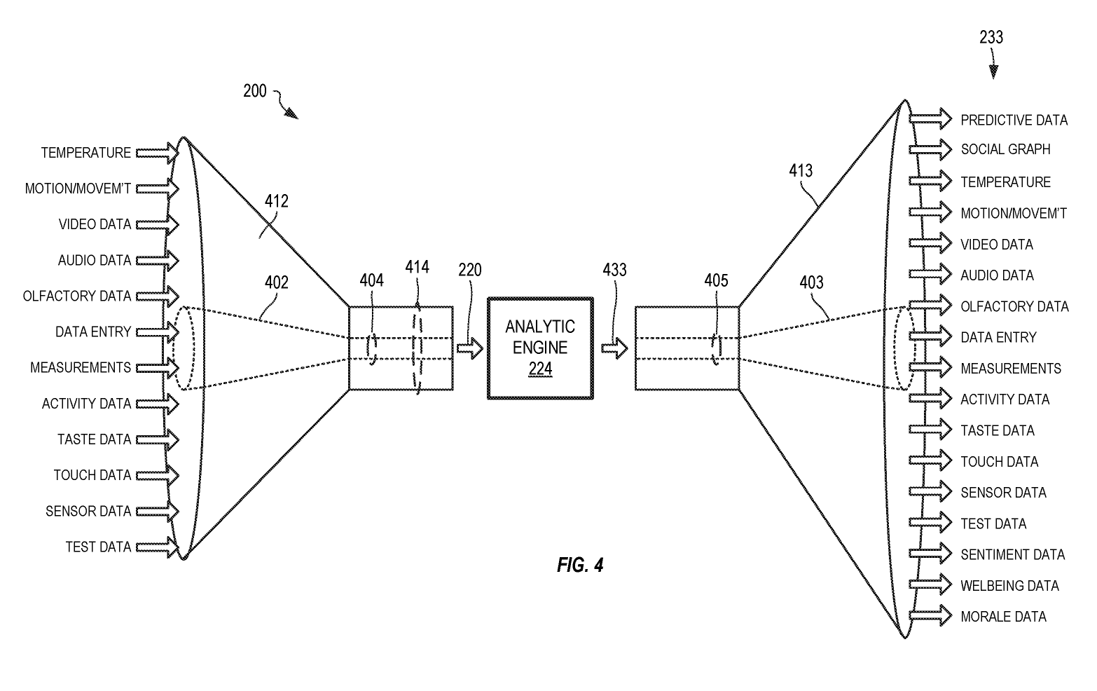

FIG. 4 is a schematic illustrating exemplary collection of healthcare information by system 200. As noted above, prior art systems collect only a small portion of available healthcare information, as indicated by dotted cone 402, resulting in a small amount 404 of useful information that was collected and made available for further processing and output, as indicated by data 405 and dotted cone 403. As shown in FIG. 1, prior art systems collect only measurements and manually entered data. System 200, on the other hand, collects quantitative data (e.g., data entry data and measurements) when available and also collects large quantities of qualitative and/or unstructured data (e.g., temperature data, motion/movement data, video data, audio data, olfactory data, activity data, taste data, touch data, sensor data and test data) as indicated by cone 412. Prior art systems are unable to use qualitative and unstructured data and therefore had no reason to collect such data. Analytic engine 224, on the other hand, processes (e.g., using NLP and other techniques described hereinbelow) qualitative and/or unstructured data such that it may be used together with quantitative data. Sensor data may represent any type of sensed information of patient 201 and test data represents the results of processed tests performed on or for patient 201.

Accordingly, system 200 still facilitates manual data entry and measurements (404) but further operates to collect temperature data, motion/movement data, video data, audio data, olfactory data, temperature data, activity data, taste data, touch data, sensor data and test data, which results in significantly larger quantity 414 of input data 220 being stored within analytic engine 224. As shown in FIG. 2, system 200 collects this data from disparate sources, and not just the doctor's consulting room. This data may include one or more of asked data, evoked data, detected data, symptom data, sign data, lab data, imaging data, test data, and sensory data. As described further below, system 200 may also collect input data 220 from social media 212, hospital 206, pharmacy 210, laboratory 208, conventional medical databases 120, and any other location where healthcare is provided to and collected from patient 201. In an embodiment, social media 212 includes data from activity and fitness tracking devices worn by patient 201.

Transducers 231 operate to collect medical information 324 such that minimal information about patient 201 is lost. This may in addition alleviate doctor 205 from the burden of interacting with a computer terminal to enter significant amounts of data, though doctor 205 may still enter notes regarding observations, diagnosis, treatment and care of patient 201. However, since transducers 231 operate to collect medical information 324 for patient 201 from patient location 204, this input data 220 may contain significantly more information that doctor 205 has time to enter manually. Further, transducer 231(2) collects medical information from within an office 203 of doctor 205, for example allowing doctor 205 to dictate additional information and thoughts on patient 201 after the consultation, scan hand written notes on patient 201, and input other relevant medical information of patient 201.

Transducer 231(3) is configured to capture medical information 324 from conventional medical database 120. For example, transducer 231(3) may be configured with or couple to conventional medical database 120 to process EMRs 121 associated with patient 201, thereby collecting historical medical information on patient 201. Transducer 231(4) is configured to collect input data 220 from within a laboratory 208. For example, as a technician tests a sample from patient 201, results of the test and details on the procedure are captured within medical information 324.

Transducer 231(5) is located within a pharmacy and operates to collect medical information 324 of patient 201. For example, transducer 231(5) may generate medical information 324 when pharmacy 210 fulfills a prescription for patient 201, and when patient 201 collects the prescription and/or purchases medications and products. Transducer 231(5) may also generate medical information 324 from conversations between a pharmacist at pharmacy 210 and patient 201. Within a hospital 206, transducer 231(6) operates to collect medical information 324 during a visit of patient 201. For example, transducer 231(6) may collect medical information 324 resulting from procedures performed on patient 201 and from interaction by patient 201 with nurses and doctors at hospital 201 during a stay by patient 201.

Transducer 231(7) is configured to collect medical information 324 from social media 212 of patient 201. For example, transducer 231(7) may generate medical information 324 from posts and tweets made by patient 201. Similarly, where patient 201 wears a tracking type device 219 that collects movement and other medical related information of patient 201, transducer 231(7) interacts with a corresponding account in social media 212 and generates medical information 324. Device 219 may also represent a portable medical device that periodically measures blood pressure of patient 201 within a defined period, wherein one or more transducers 231 wirelessly connect to device 219 to collect the measured data.

Analytic engine 224 stores and processes input data 220 and generates one or more medical intensity status displays 233. In the example of FIG. 2, system 200 generates medical intensity status display 233(1) within doctor's office 203. However, medical intensity status display 233(1) may be provided at any desired location, such as to doctor 205 during a consultation with patient 201 at patient location 204. Medical intensity status display 233 provides an enhanced view of the health of patient 201 and may indicate predicted medical events for patient 201.

Analytic engine 224 is a big data analytical engine for processing input data 220 to infer one or more of patient sentiment, patient general wellbeing, patient morale, patient activity, and social graph. In the embodiments herein, sentiment is the meaning, context, conveyed message and impression. As shown in FIG. 4, analytic engine 224 generates a patient medical model 433 that defines past health events and current health status of patient 201, and predicts future health events of patient 201. As shown, patient medical model 433 defines many healthcare aspects of patient 201 and may be used to generate medical intensity status display 233 to include one or more of video data, audio data, olfactory data, data entry, measurement values, activity data, taste data, touch data, predictive data, social graph, sentiment data, wellbeing data, and moral data. That is, analytic engine 224 generates medical intensity status display 233 to provide a more complete health status of patient 201 that was previously possible. Further, analytic engine 224 may operate continuously and/or periodically to continuously update knowledgebase 226.

System 200 also integrates with the larger EHR. For example, information the collected by transducers 231, as described above, may be displayable within the EHR display (e.g., EPIC or CERNER) and/or other similar constructs and systems. Certain of this collected information may be discoverable and analyzable via “Big Data” tools and systems as described in Appendix A and Appendix B of U.S. patent application Ser. No. 62/194,904.

Context

Transducer 231(1) also provides context to the consultation between doctor 205 and patient 201. For example, where patient 201 is an elderly parent accompanied by a child, the behavior of patient 201, and information supplied by patient 201, may differ from behavior and supplied information when patient 201 visits doctor 2005 unaccompanied. Other transducers 231 may provide context to collected input data 220 at other locations. That is, input data 220 includes context information for patient 201 based upon information collected from other people in proximity to the patient. Not only information as to who was present at the gathering, but also sentiment of those people as they may also affect patient 201. Information from one gathering where another person was present may also be correlated to other gatherings having the same person present, since presence of that person may skew information collected from patient 201. For example, where sentiment of patient 201 changes when the other person arrives, analytic engine 224 may determine that the other person invokes anxiety within patient 201.

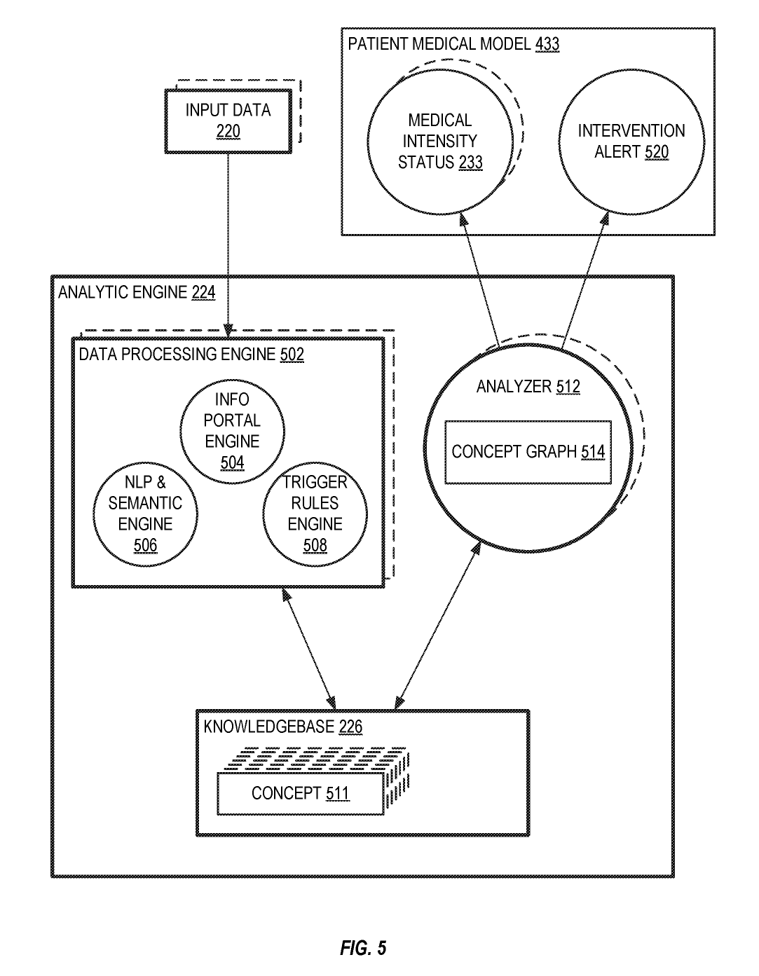

FIG. 5 shows analytic engine 224 in exemplary detail, including at least one data processing engine 502 that processes received input data 220 to create knowledgebase 226. Analytical engine 224 is described in greater detail within Appendix A and Appendix B of U.S. patent application Ser. No. 62/194,904. Therefore, analytic engine 224 will be discussed only briefly herein. Data processing engine 502 has an information portal engine 504 that uses a trigger rules engine 508 and a NLP and semantic engine 506 to process input data 220 to determine healthcare concepts 511 for storage within knowledgebase 226. As shown in FIG. 2, input data 220 is received from a plurality of disparate sources and may include audio, images, hand written notes, raw data, test results, and the like. Information portal engine 504 uses trigger rules engine 508 to identify language elements within input data 220 that correspond to healthcare data of interest. Healthcare data of interest may include one or more of asked data, evoked data, detected data, symptom data, sign data, lab data, imaging data, test data, and sensory data. Further, information portal engine 504 uses NLP and semantic engine 506 to discern healthcare data of interest from input data 220 derived from language used by people (e.g., patient 201, doctor 205, and so on). Specifically, NLP and semantic engine 506 identifies semantic relationships between identified concepts 511 within input data 220, such that healthcare data concepts 511 are stored within knowledgebase 226 together with their relationship to one another.

In an embodiment, analytic engine 224 also includes an analyzer 512 that utilizes selected healthcare concepts 511 from knowledgebase 226 to generate a concept graph 514 associated with patient 201. Analyzer 512 uses concept graph 514 to generate patient medical model 433 for patient 201. Patient medical model 433 may be considered a virtual reality that defines the status of patient 201 within analytic engine 224. Patient medical model 433 is based upon all collected input data 220 for patient 201, including audio data, video data, medical records, test results, and so on, where analytic engine 224 correlates all collected data to form a comprehensive healthcare model of patient 201. For example, analytic engine 224 correlates sentiment, test results, and healthcare information derived from multiple sources of input data 220 and stored within knowledgebase 226 to form patient medical model 433. Knowledgebase 226 is continually and/or periodically updated such that knowledgebase 226 grows to contain large quantities (big data) of healthcare data.

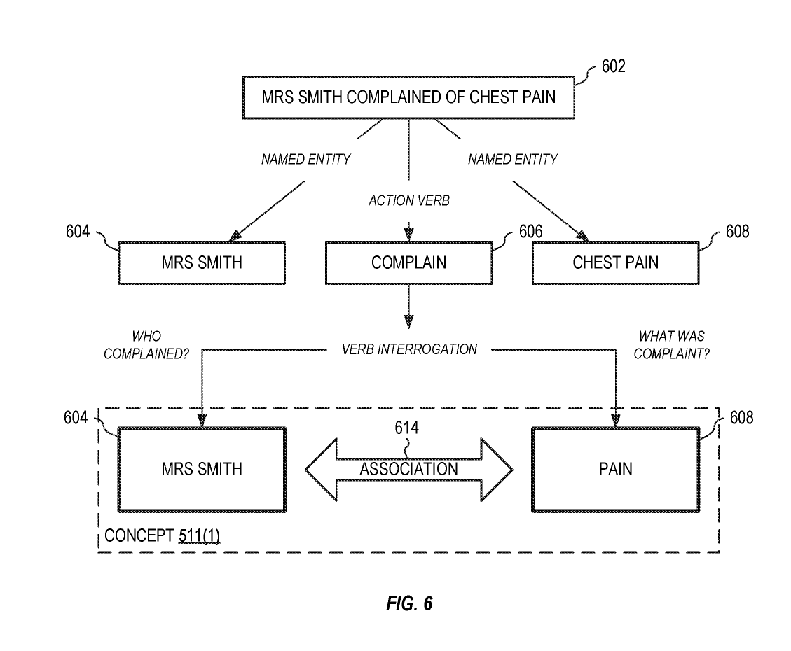

FIG. 6 is a schematic illustrating exemplary analysis of a natural language phrase 602 to identify concept 511. Phrase 602 is for example received as notes made by doctor 205 (for example in data entry 402, FIG. 4). Information portal engine 504 uses NLP and semantic engine 506 to identify named entity 604, action verb 606, and second named entity 608 within phrase 602. NLP and semantic engine 506 then, based upon verb interrogation, forms a complaint concept 511(1) to associate, indicated by arrow 614, named entity 604 (Mrs. Smith) with second named entity 608 (Pain). Syntactic variations in natural language may also be mapped together. Exemplary syntactic variations for the verb “to complain” may for example include: “complained”, “has complained”, “is complaining”, “will complain”, “which complained”, “is not complaining”, “could have complained”, “shall not complain”, “will not complain”, and so on. Thus, the verb may be formed of a one word tuple (e.g., “complained”), of a two word tuple (e.g., “has complained”), and a three word tuple (e.g., “could have complained”).

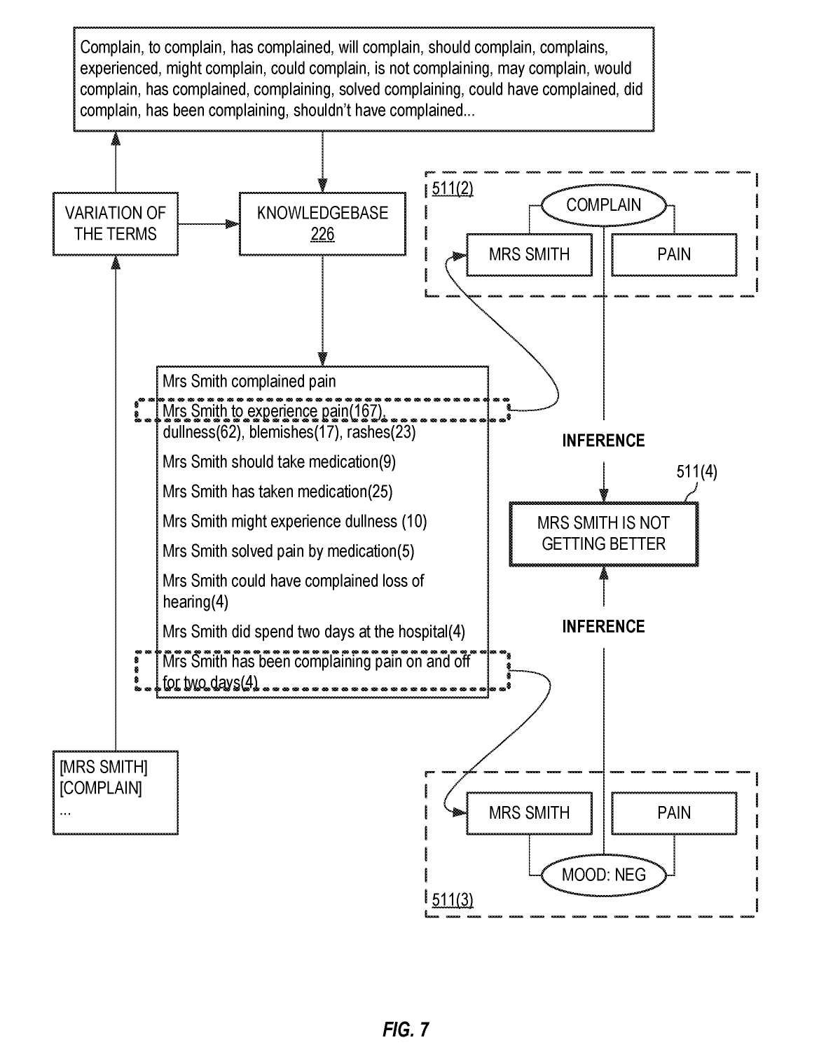

FIG. 7 is a schematic illustrating exemplary inference, by analyzer 512 of FIG. 5, of one concept 511(4) from two other concepts 511(2) and 511(3). Concept 511(2) includes information that Mrs. Smith complained of pain, and concept 511(3), which occurred at a later time than information used to determine concept 511(2), includes information that Mrs. Smith has a negative mood because of intermittent pain over two days. Based upon concepts 512(2) and 512(3), analyzer 512 automatically infers concept 511(4) that includes information that Mrs. Smith is not getting better. Inferred concept 511(4) is also stored within knowledgebase 226.

Analyzer 512 may corroborate and reinforce the accuracy of inferences derived from concepts 511, 512 using contemporaneously measured variables. For example, where data collected from sensor readings and/or direct examination data indicate that Mrs. Smith has an increase in heart rate and/or blood pressure and sweating, which are typical symptoms of a patient in pain, analyzer 512 reinforces the inference that Mrs. Smith is not getting better.

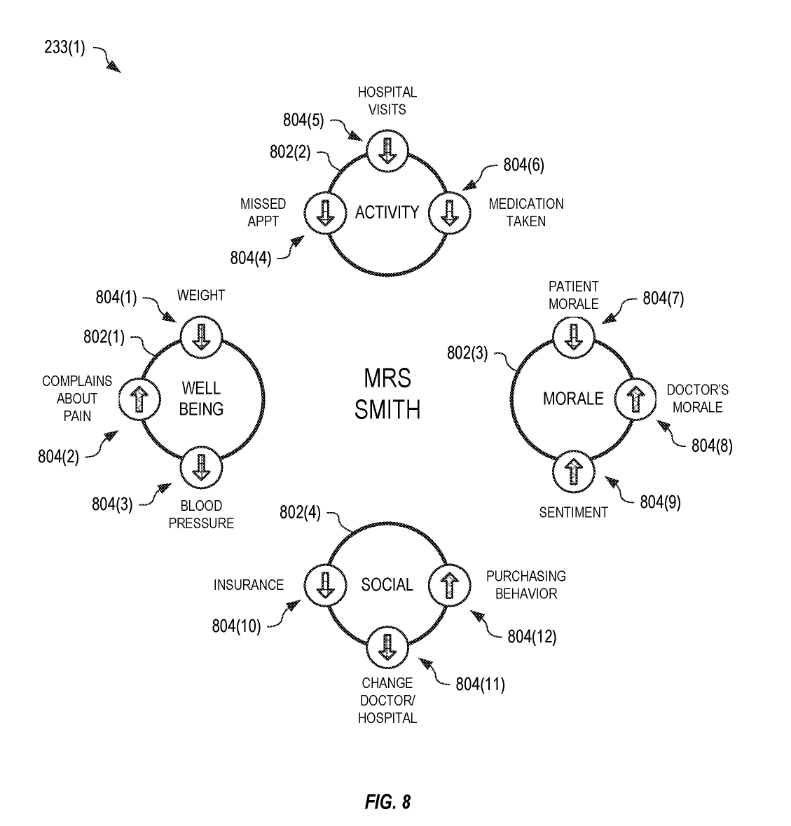

FIG. 8 is a schematic showing one exemplary medical intensity status display 233(1), FIG. 2, generated from patient medical model 433, FIG. 4, for Mrs. Smith. Patient medical model 433 is derived by analyzer 512 from concepts 511 stored within knowledgebase 226. By creating and maintaining knowledgebase 226 of concepts 511 determined from input data 220, and by deriving additional concepts 511 from concepts 511 stored within knowledgebase 226, system 200 generates patient medical model 433 that may in turn be used to generate medical intensity status display 233 to provide a more complete knowledge of patient 201 to doctor 205 during the consultation with patient 201. Patient medical model 433 allows system 200 to generate medical intensity display 233(1) that contains more detail regarding the health of patient 201 than is currently available using prior art medical data analysis systems.

In FIG. 8, medical intensity status display 233(1) is illustratively shown with four status areas: wellbeing 802(1), activity 802(2), morale 802(3), and social 802(4). However, medical intensity status display 233 may have more or fewer status areas 802 without departing from the scope hereof. Each status area 802 illustrates three exemplary trends 802, where each trend includes an arrow that indicates change in the trend. In one embodiment, the size of the arrow is proportional to the magnitude of the change in the trend. In another embodiment, a color of the arrow indicates whether the trend is good (e.g., green) or bad (e.g., red). Wellbeing 802(1) shows weight 804(1), complaints about pain 804(2), and blood pressure 804(3), activity 802(2) shows missed appointments 804(4), hospital visits 804(5), and medication taken 804(6), morale 802(3) shows patient morale 804(7), doctor morale 804(8), and sentiment 804(9), and social 802(4) shows insurance 804(10), change doctor/hospital 804(11), and purchasing behavior 804(12). Each status area 802 may have more or fewer trends 804 without departing from the scope hereof. Trends 804 may be automatically selected for each status area 802, or may be manually selected by interacting with system 200.

By viewing medical status intensity 233(1), FIG. 2, while consulting with patient 201, doctor 205 is better informed as to the current health status of patient 201, and learns of recent trends 804 in the health and behavior of patient 201.

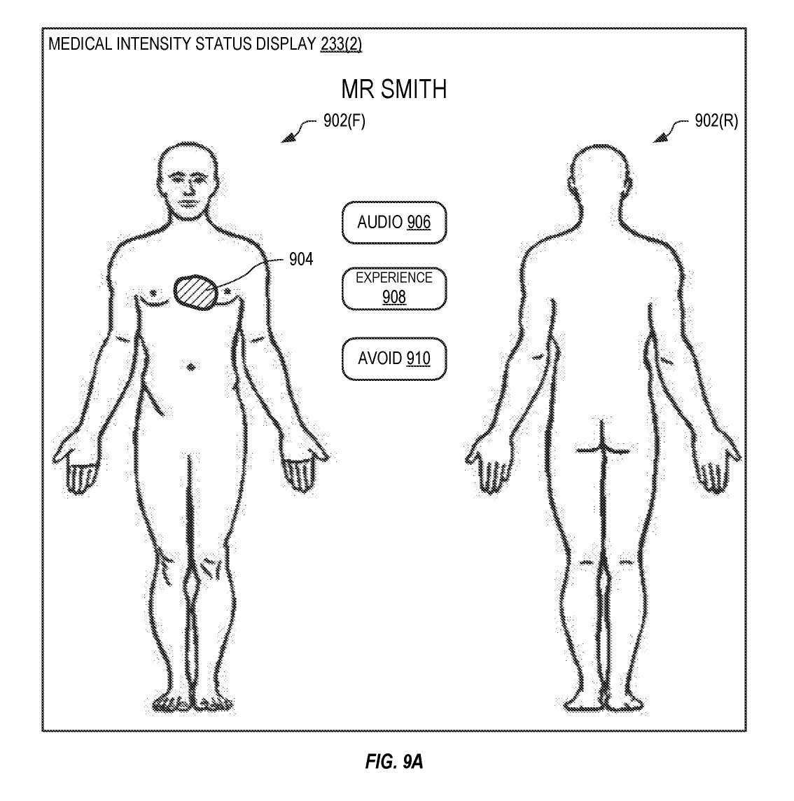

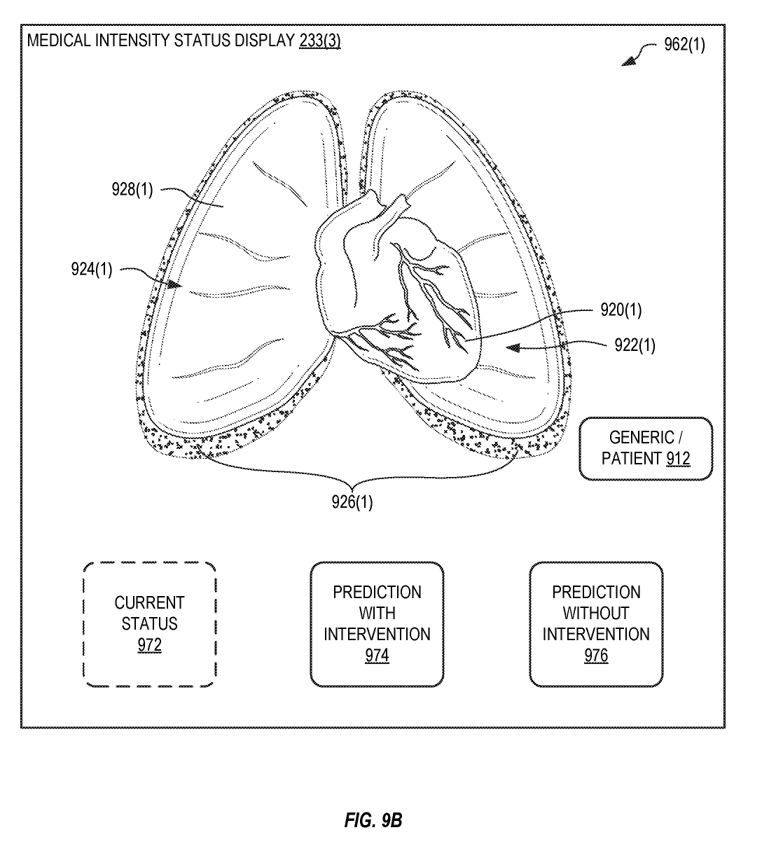

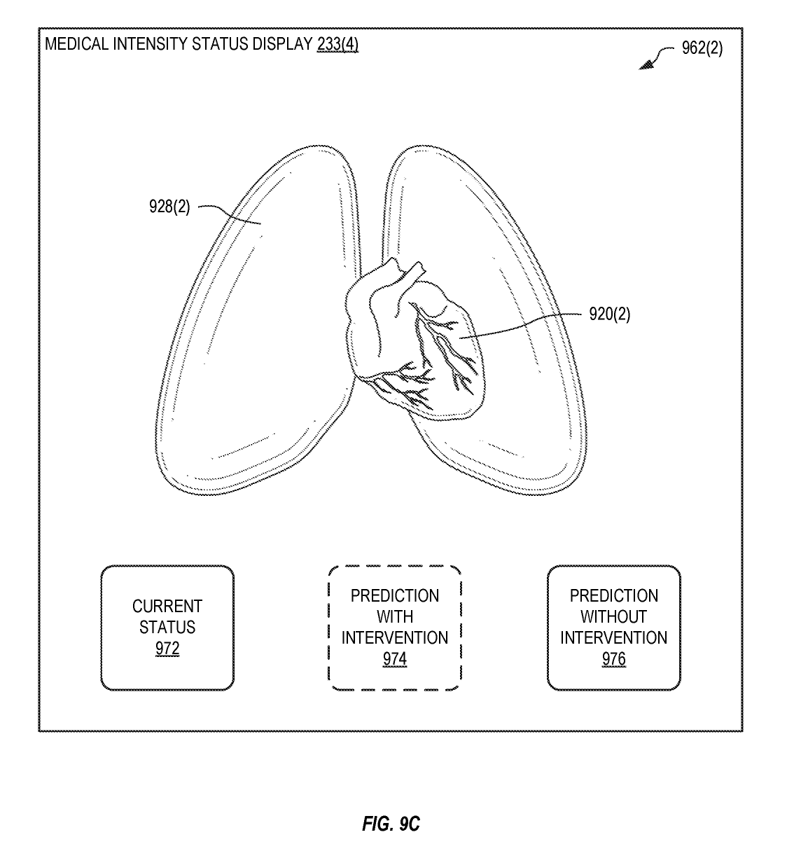

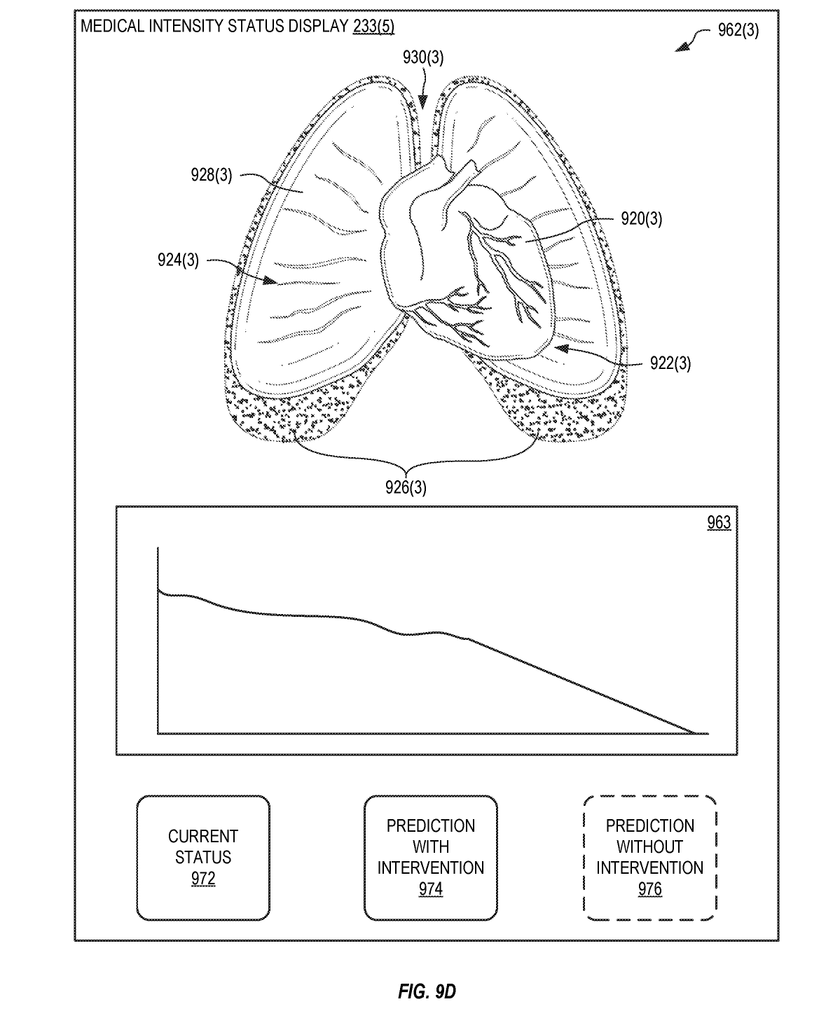

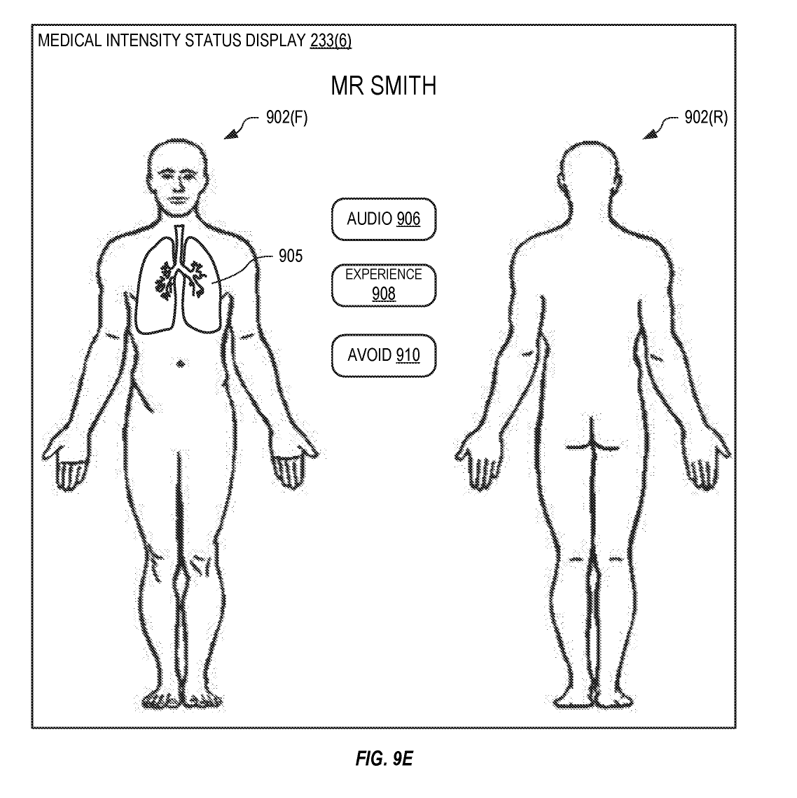

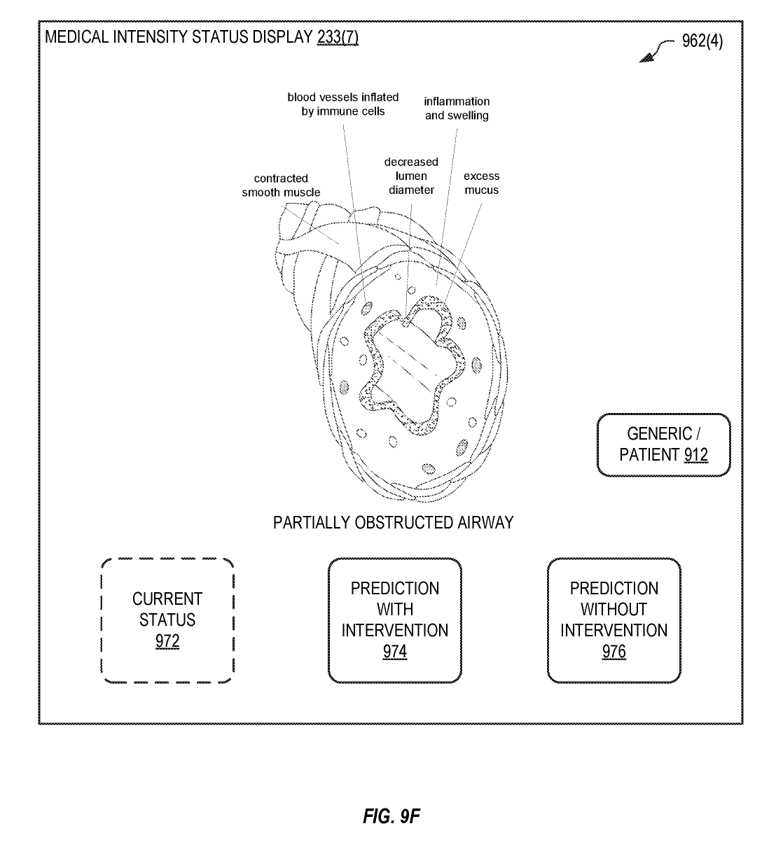

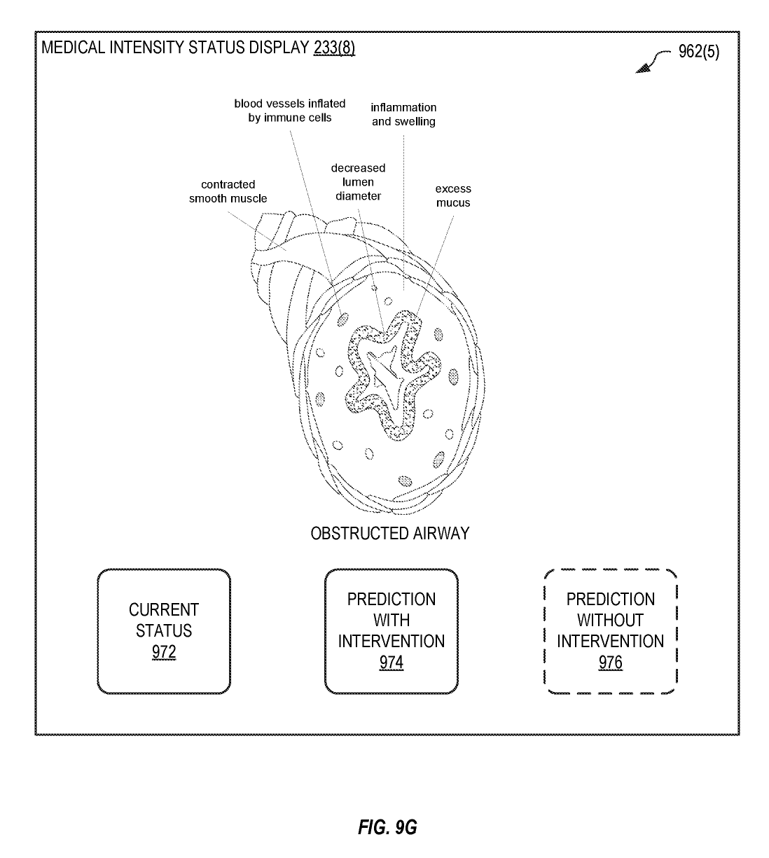

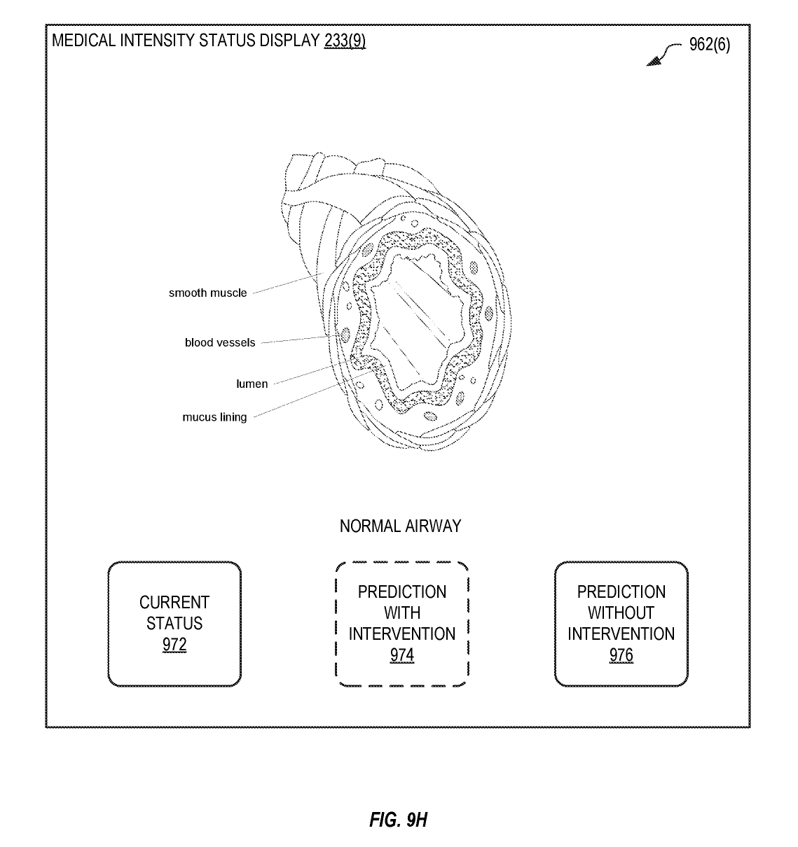

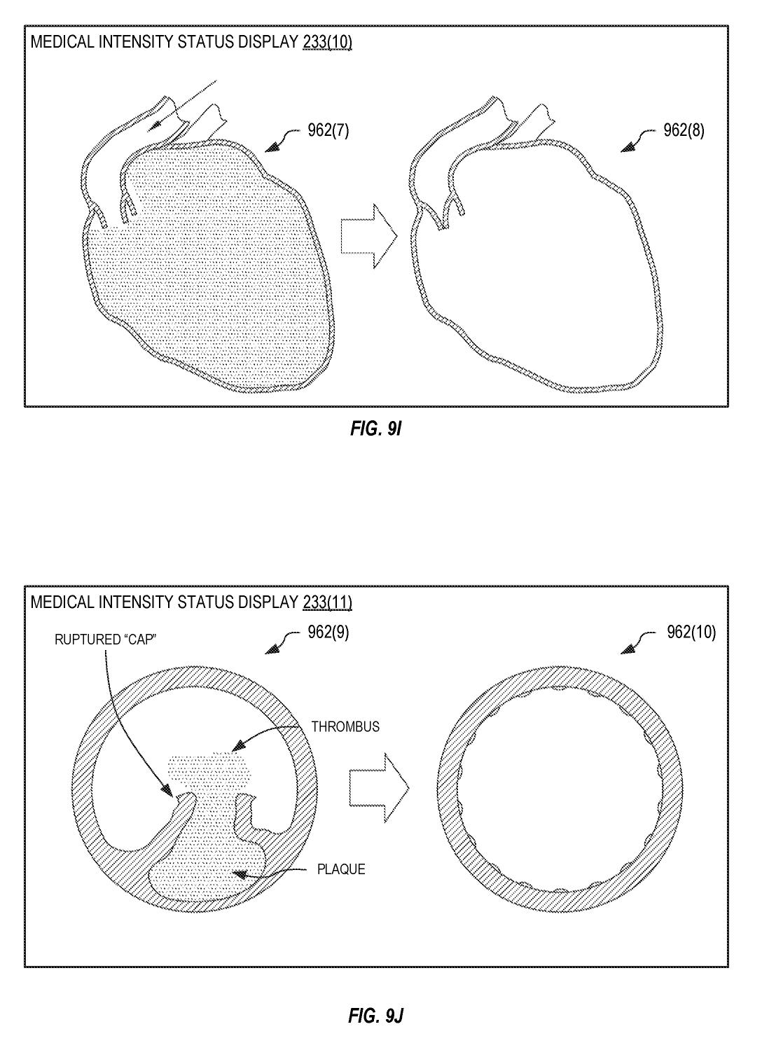

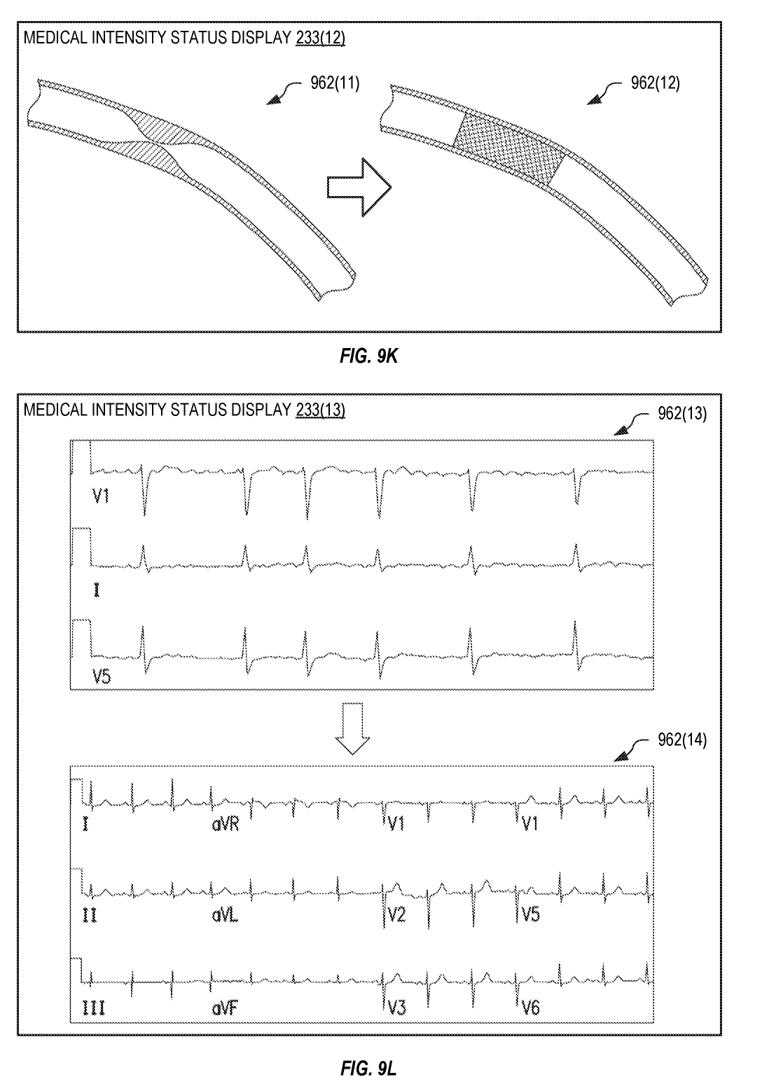

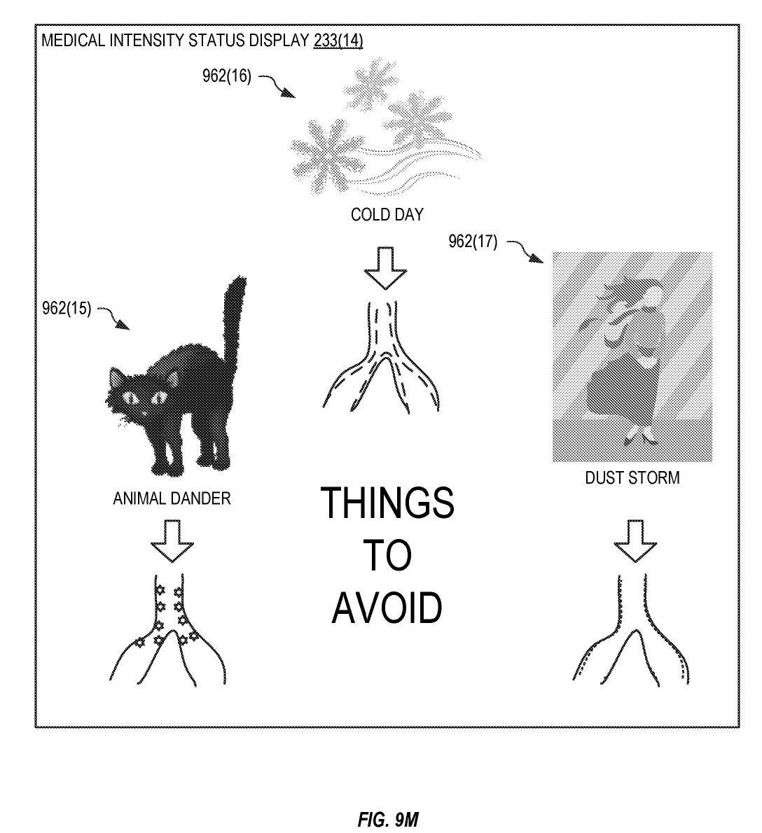

FIG. 9A is a schematic showing one exemplary medical intensity status display 233(2) generated from patient medical model 433, FIG. 5, for patient 201 (Mr. Smith) who has heart disease. Medical intensity status display 233(2) shows a front (F) and a rear (R) anatomical model 902 with a highlighted heart 904, indicating the current medical problem of patient 201. Medical intensity status display 233(2) may include an audio button 906 that, when selected, plays audio of her heartbeat. This audio may be a playback of a previous recording of the actual heart of patient 201, or it may be a generic audio recording or simulation of heart disease. Using intensity display 233(2), doctor 205 better illustrates the problem to patient 201, and patient 201 gains a better understanding of the problem as it specifically pertains to him/her. For example, patient medical model 433 is configured with the most accurate rendition of the medical issue facing patient 201 based upon collected healthcare data of patient 201. Medical intensity status display 233(2) may also include an experience button 908 that, when selected, illustrates effects of the disease that patient 201 may yet come to experience. For example, where patient 201 has an early diagnosis of heart disease, doctor 205 may select button 908 to display future exemplary symptoms that may be experienced by patient 205. For example, for a patient with heart failure medical intensity status display 233(2) may show anatomic depictions—either actual or stylized—for patient 201. Images that may be displayed include: Chest X-ray, 2D or 3D echo, trans esophageal echo, CT scan—e.g. Ultrafast CT, or MRI/MRA images. Medical intensity status display 233(2) may show images that are either actual or may be modifiable so that the health worker (MD) may alter the time course—e.g. accelerate or decelerate the disease process—with or without therapy to illustrate to the patient the importance of a therapy and the consequences of non-compliance, etc. Medical intensity status display 233(2) may also show, and/or reproduce, additional symptoms and signs as well as allow the patient to experience the clinical scenario for better learning and appreciation.

In one embodiment, anatomical model 902 is personalized (e.g., facial image, body color, shape and size, and so on) such that patient 201 is more aware that the displayed medical information specifically relates to him/her, and thereby better assimilates and retains the provided information.

FIG. 9B shows one exemplary medical intensity status display 233(3) resulting from selection of the displayed heart 904 in medical intensity status display 233(2) of FIG. 9A. Medical intensity status display 233(3) includes a graphic 962(1) showing detail of the current status of the medical problem with the heart of patient 201. In this example, graphic 962(1) is an X-ray image showing that patient 201 is approaching systolic heart failure. In particular, graphic 962(1) shows enlargement 922(1) of heart 920(1), indications of Kerley “B” lines 924(1) in lungs 928(1), and pleural effusions 926(1). By displaying medical intensity status display 233(3), doctor 205 is better able to educate patient 201 of her current medical problem. Graphic 962(1) may or may not be animated. Medical intensity status display 233(3) also includes a prediction with intervention button 974 and a prediction without intervention button 976. Since medical intensity status display 233(3) shows the current status, a current status button 972 is non-selectable (e.g., greyed out). Medical intensity status display 233(3) may also include a generic/patient button 912 that allows the display to be toggled between a generic view of the displayed disease and a patient related view of the displayed disease that shows the disease based upon healthcare data of patient 201.

FIG. 9C shows one exemplary medical intensity status display 233(4) resulting from selection of prediction with intervention button 974 in medical intensity status display 233(3) of FIG. 9B. Medical intensity status display 233(4) includes a graphic 962(2) showing a predicted state of the heart of patient 201 based upon patient 201 taking intervention. Graphic 962(2) is for example an X-ray type image. In the example of FIG. 9C, heart 920(2) is shown normal size, and lungs 928(2) are clear. Patient medical model 433 may include information pertaining to results of other patients having similar conditions that followed a prescribed intervention.

FIG. 9D shows one exemplary medical intensity status display 233(5) resulting from selection of prediction without intervention button 976 in medical intensity status display 233(3) of FIG. 9B (or from medical intensity status display 233(4) of FIG. 9C). Medical intensity status display 233(5) includes a graphic 962(3) showing a state of the heart of patient 201 that is predicted based upon patient 201 not taking intervention. In this example, graphic 962(3) is an X-ray type image showing that patient 201 has systolic heart failure. In particular, graphic 962(3) shows enlargement 922(3) of heart 920(3), clear Kerley “B” lines 924(3) in lungs 928(3), pleural effusions 926(3), and cephalization of flow 930(3). For example, patient medical model 433 includes information pertaining to results of other patients having similar conditions that did not follow any prescribed intervention. Where patient 201 fails to comply with a prescribed intervention, medical intensity status display 233(5) may also display a progression chart 963 based upon healthcare data of patient 201 and predicted effects of the non-compliance. Chart 963 may display a predicted death of patient 201, where predicted data indicates death is likely. Such charts may therefore be a powerful tool for encouraging patient 201 to comply with the prescribed intervention.

Doctor 205 may show one or both of medical intensity status display 233(4) and medical intensity status display 233(5) to patient 201 to better illustrate use of prescribed interventions and to illustrate what happens from not following prescribed interventions. Since medical intensity status display 233(5) is based specifically upon healthcare information of patient 201, the predictions of medical intensity status display 233(4) and medical intensity status display 233(5) have a high accuracy probability and may therefore have more impact upon patient 205, particularly when patient 201 is not following a prescribed intervention (e.g., taking a prescribed drug).

FIG. 9E is a schematic showing one exemplary medical intensity status display 233(6) generated from patient medical model 433, FIG. 5, for patient 201 (Mr. Smith) who has Asthma. Medical intensity status display 233(6) is similar to medical intensity status display 233(3) of FIG. 9A, showing a front (F) and a rear (R) anatomical model 902. However, in the example of FIG. 9E, lungs 905 are highlighted to illustrate that patient 201 is suffering from Asthma. In the example of FIG. 9E, when audio button 906 is selected, the wheezing sound of constricted breathing may be heard. This sound may be generated from a recording of breathing by patient 201, or may be a generic recording of breathing by an Asthma sufferer. Medical intensity status display 233(6) may also include an avoid button 910 that may be selected to allow doctor 205 to illustrate conditions for patient 201 to avoid. Operation of button 910 a described below with reference to FIG. 9M.

FIG. 9F shows one exemplary medical intensity status display 233(7) resulting from selection of the displayed lungs 905 in medical intensity status display 233(6) of FIG. 9E. Medical intensity status display 233(7) includes a graphic 962(4) showing detail of a current condition of bronchial tubes of patient 201. By displaying medical intensity status display 233(7), doctor 205 is better able to educate patient 201 of his current medical problem. Graphic 962(4) may or may not be animated. Medical intensity status display 233(7) also includes prediction with intervention button 974 and prediction without intervention button 976. Since medical intensity status display 233(7) shows the current status, current status button 972 is non-selectable (e.g., greyed out).

For example, with asthma and wheezing, the sound/symptom may be reproduced with an effector that allows patient 201 to “feel” the symptom/signs as a vibration or other somato sensory experience, further imprinting and enhancing learning for the patient.

FIG. 9G shows one exemplary medical intensity status display 233(8) resulting from selection of prediction with intervention button 974 in medical intensity status display 233(7) of FIG. 9F. Medical intensity status display 233(8) includes a graphic 962(5) showing a state of the bronchial tubes of patient 201 that is predicted based upon patient 201 not taking intervention. For example, patient medical model 433 includes information pertaining to results of other patients having similar conditions that did not follow prescribed interventions.

FIG. 9H shows one exemplary medical intensity status display 233(9) resulting from selection of prediction without intervention button 976 in medical intensity status display 233(7) of FIG. 9F (or from medical intensity status display 233(8) of FIG. 9G). Medical intensity status display 233(9) includes a graphic 962(6) showing a state of the bronchial tubes of patient 201 that is predicted based upon patient 201 taking intervention. For example, patient medical model 433 includes information pertaining to results of other patients having similar conditions that followed prescribed interventions.

FIG. 9I shows one exemplary medical intensity status display 233(10) that includes an exemplary graphic 962(7) illustrating aortic insufficiency with decompensation, and an exemplary graphic 962(8) illustrating medical or surgical therapy of the aortic insufficiency.

FIG. 9J shows one exemplary medical intensity status display 233(11) that includes an exemplary graphic 962(9) illustrating a vulnerable plaque rupture, as occurs with acute contrary syndrome (or MI), and an exemplary graphic 962(10) illustrating s/p stenting with intravascular ultrasound to correct the vulnerable plaque rupture, such that the artery is open, sealed, and supported.

FIG. 9K shows one exemplary medical intensity status display 233(12) that includes an exemplary graphic 962(11) illustrating an angiogram of hi-grade coronary artery disease, and an exemplary graphic 962(12) illustrating s/p stenting to correct the artery disease.

FIG. 9L shows one exemplary medical intensity status display 233(13) that includes an exemplary graphic 962(13) illustrating a ECG strip of a heart in Afib, and an exemplary graphic 962(14) illustrating an ECG strip of the heart after Afib correction.

FIG. 9M shows one exemplary medical intensity status display 233(14) resulting from selection of avoid button 910 in medical intensity status display 233(6) of FIG. 9E. Medical intensity status display 233(14) shown one or more examples of that patient 201 should try to avoid to prevent further complication of current medical issues. Continuing with the current example, medical intensity status display 233(14) shows a graphic 962(15) illustrating the effect of animal dander on bronchial tubes of patient 201, a graphic 962(16) showing the effects of cold air on bronchial tubes of patient 201, and a graphic 962(17) showing the effects of dust on bronchial tubes of patient 201. Using medical intensity status display 233(14), doctor 205 may better reinforce the benefits of avoiding certain conditions for patient 201, as compared to providing only verbal instruction.

In another example, analyzer 512 infers patient 201 has anorexia nervosa based upon concepts 511 and 512. System 200 generates medical intensity status display 233 to show a healthy individual of similar height and characteristics to the patient and the corresponding caloric intake required to sustain that physique. System 200 may then generate medical intensity status display 233 to show the patient's current state as being under weight and low body mass index for comparison. System 200 then generates medical intensity status display 233 to illustrate the effects of further caloric deprivation, such as muscle withering, skin degeneration, hair degeneration and loss, reproductive organ damage, menstrual cycle changes, and mood changes. System 200 then generates medical intensity status display 233 to illustrate the possibility of repair to the patient's body by increasing caloric intake.

The prediction derived from patient medical model 433 may also be used to evaluate certain interventions for a given diagnosis. For example, doctor 205 may run patent medical model 433 to determine an effect of a proposed intervention on patient 201 based upon the actual effect of the intervention upon patient having similar conditions to patient 201. That is, analytic engine 224 may be used to predict the effect of certain interventions upon patient 201 before they are prescribed, thereby reducing the probability of prescribing a treatment that is not optimal.

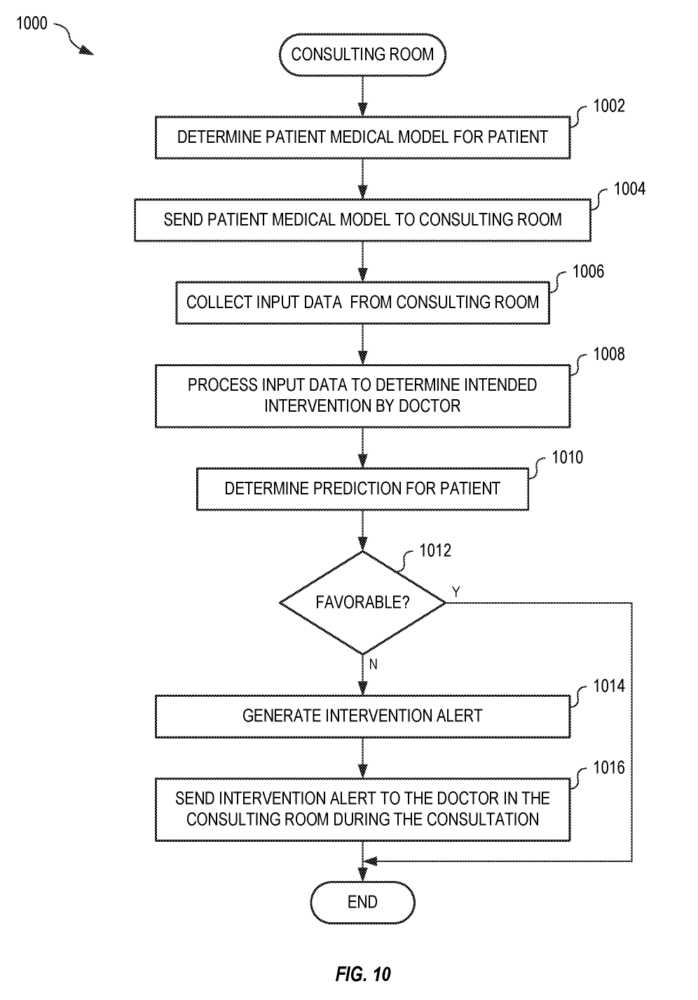

FIG. 10 shows one exemplary medical feedback continuum method 1000. Method 1000 is for example implemented within system 200 of FIG. 2 and operates when patient 201 is consulting with doctor 205 at patient location 204. In particular, method 1000 uses analytic engine 224 to generate alerts when a suggested intervention is not optimal for patient 201.

In step 1002, method 1000 determines medical intensity status of the patient. In one example of step 1002, analyzer 512 determines medical intensity status 233 of patient 201 based upon concept graph 514 constructed from selected concepts 511 of knowledgebase 226. In step 1004, method 1000 sends medical intensity status to the consulting room. In one example of step 1004, analyzer 512 sends medical intensity status 233 to doctor's office 203 for display to doctor 205.

In step 1006, method 1000 receives input data for patient from consulting room. In one example of step 1006, transducer 231(1) collects input data 220 from patient location 204. In step 1008, method 1000 processes input data to determine intended intervention by doctor. In one example of step 1008, where patient location is a consulting room of doctor 205, algorithms 320 within transducer 231(1) and data processing engine 502 cooperate to understand natural language within input data 220 collected from patient location 204 and determine a diagnosis made by doctor 205 and an intended intervention for patient 201 prescribed by doctor 205.

In step 1010, method 1000 determines a prediction for the patient. In one example of step 1010, analyzer 512 constructs concept graph 514 from knowledgebase 511 and determines a probably medical outcome from the intended intervention for patient 201.

Step 1012 is a decision. If, in step 1012, method 1000 determines that the intended intervention and predicted medical outcome are favorable, method 1000 goes back to step1006 and repeats; otherwise, method 1000 continues with step 1014. In step 1014, method 1000 generates an intervention alert for the patient. In one example of step 1014, analyzer 512 generates an intervention alert 520 indicating the determined probable medical outcome. In step 1016, method 1000 sends the intervention alert to the consulting room. In one example of step 1016, analyzer 512 sends intervention alert 520 to doctor's office 203 for display to doctor 205.

Method 1000 may also apply to discharge of patient 201 from hospital 206, wherein system 200 invoked analyzer 512 to predict a medical outcome for patient 201, and may generate an intervention alert 520 if patient is being discharged from hospital but is predicted to return.

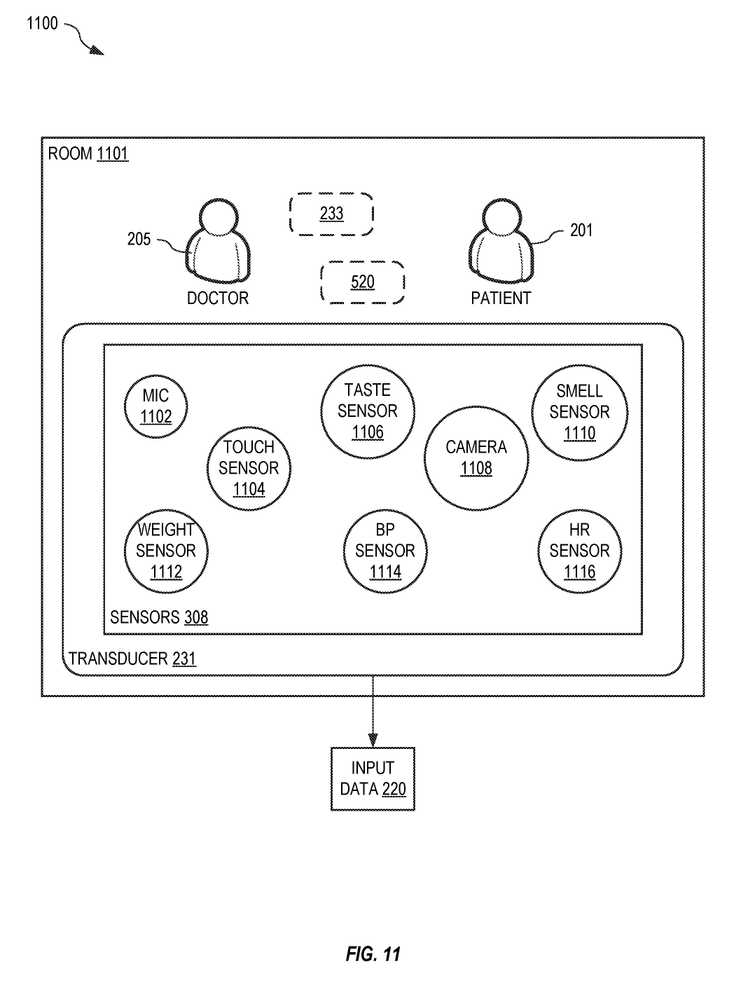

FIG. 11 shows exemplary sensors of transducer 231 of FIG. 2 used within a room 1101. Room 1101 may represent patient location 204, such as a consulting room, a hospital room, or other such places where patient 201 may have a medical encounter, such as consulting with doctor 205 and/or other medical providers, or otherwise be seen, examined, and/or interacted with. Transducer 231 may include, for example, a microphone 1102 for detecting audio within room 1101, a touch sensor 1104 to detecting pressure and/or texture of patient 201, a taste sensor 1106 for tasting one or more samples from patient 201, a camera 1108 for capturing one or more (still or moving) images of patient 201, a smell sensor 1110 for detecting smells within consulting room 1101, a weight sensor 1112 for detecting weight of patient 201, a blood-pressure sensor 1114 for determining a blood-pressure of patient 201, and a heart rate sensor 1116 for detecting a heart rate of patient 201. Transducer 231(1) may include other sensors and testing devices without departing from the scope hereof. If included, these sensors may gather additional valuable diagnostic, therapeutic and prognostic information. Advantageously, the use of sensors with system 200 allow information to be gathered rapidly, reliably, accurately, and objectively, without added burden to the otherwise time stressed health care worker (e.g., MD). Prior to system 200, this type of data was not entered into patients' charts/Computer/EHR, and was therefore lost. Information collected by system 200 improves the accuracy of medical diagnosis as well as providing certain information for patient outcome trending, for example, and for “big data” model building, to name just a few examples of use for the collected information.

To illustrate the advantages provided by system 200, consider the following scenario: a first patient has no outward appearance of difficulty but states that he/she is short of breath; a second patient has visually apparent ambulation difficulty, is clearly struggling for breath (e.g., gasping with air hunger). The traditional, prior to system 200, EHR has limited data entry locations (e.g., text data entry boxes on an electronic form) such that a doctor would likely enter “shortness of breath” for each of the first and second patients. However, in reality, the second patient has clear distress, is likely in a much worse pathophysiologic status, and has a worse prognostic status than the first patient. However, using the traditional EHR input mechanism, this differentiating information is lost, unless actively entered, via textual distinction, into the EHR. On the other hand, system 200 uses transducers 231 to capture an additional rich layer of information that may be quantified, displayed, recalled, and analyzed.

Transducer 231(1) thereby captures information based upon conditions experienced by doctor 205 within consulting room 1101. Transducer 231 may be located anywhere that patient 201 receives healthcare. For example, transducer 231 may be mobile and transported by doctor 205 during a house call to patient 201.

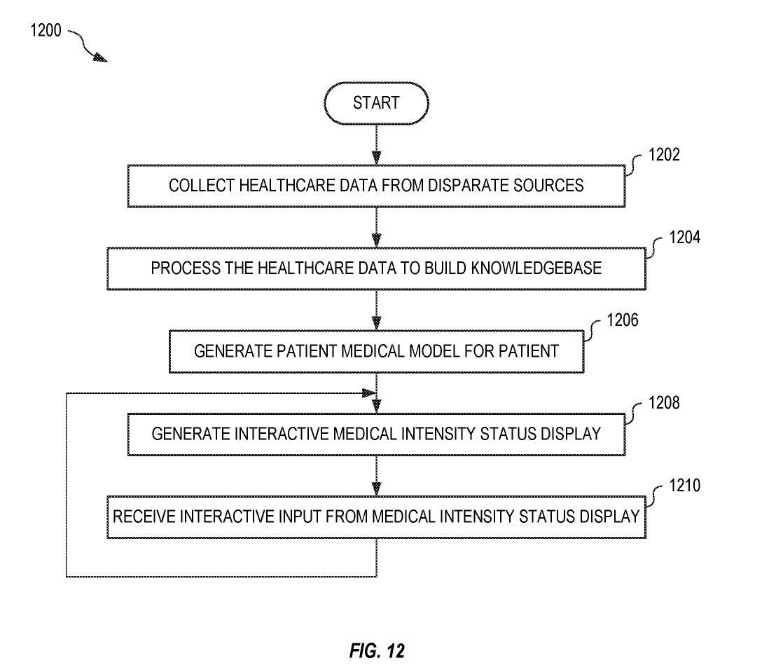

FIG. 12 is a flowchart illustrating one exemplary medical feedback continuum method 1200. Method 1200 is implemented at least part within transducers 231 of system 200, FIG. 2, and at least in part within analytic engine 224 of system 200.

In step 1202, method 1200 collects healthcare data from disparate sources. In one example of step 1202, transducers 231 collect input data 220 from disparate sources using a plurality of sensors. In step 1204, method 1200 processes the healthcare data to build a knowledgebase. In one example of step 1204, analytic engine 224 processes input data 220 and generates knowledgebase 226. In step 1206, method 1200 generates a patient medical model for a patient. In one example of step 1206, analyzer 512 within analytic engine 224 generates patient medical model 433 from knowledgebase 226. In step 1218, method 1200 generates an interactive medical intensity status display. In one example of step 1208, system 200 generates medical intensity status display 233 from patient medical model 433 at patient location 204. In step 1210, method 1200 receives interactive input from the medical intensity status display. In one example of step 1210, analytic engine 224 receives input from medical intensity status display 233 resulting from interaction by doctor 205 within doctors' office 203.

Steps 1208 through 1210 repeat to allow doctor 205 to interactively educate patient 201 at patient location 204.

Example Implementation

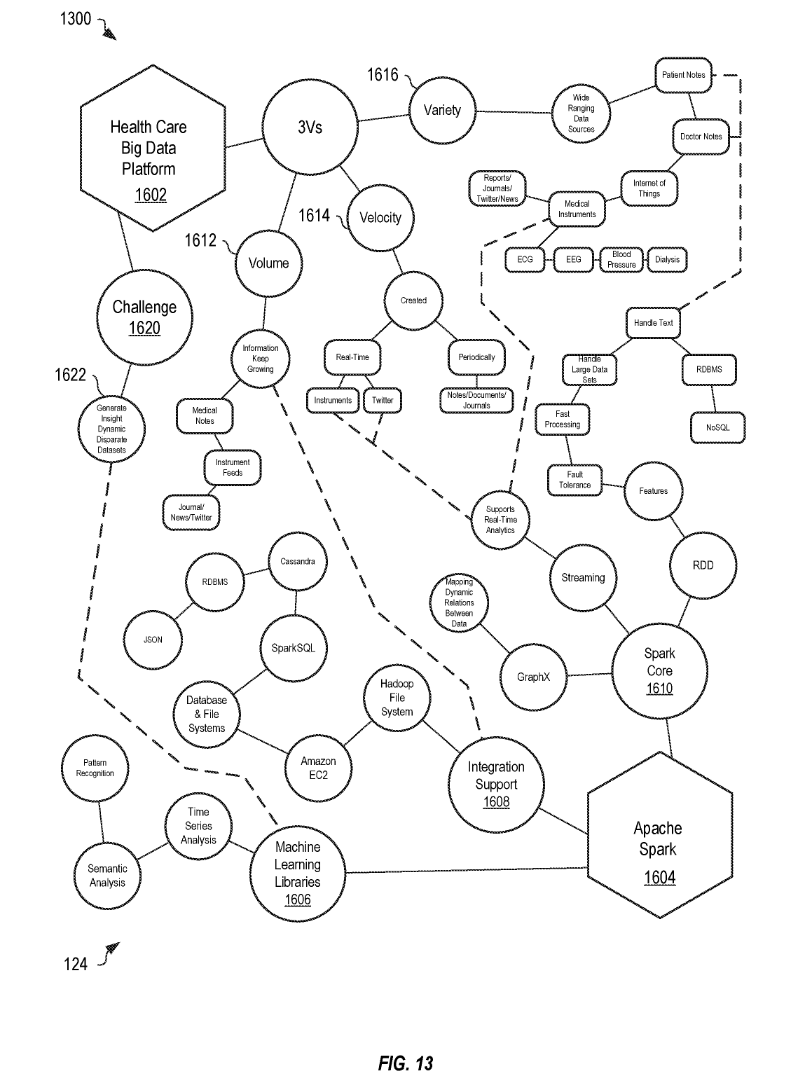

FIG. 13 shows one exemplary framework 1300 for implementing analytic engine 224 of FIGS. 2, 4, and 5 using an Apache Spark platform, in an embodiment. Framework 1300 depicts health care big data's 3Vs and expands them with health care examples.

A healthcare big-data platform 1302 is shown at the top left of framework 1300 and a ‘generic’ Apache Spark 1304 is shown at the bottom right. Framework 1300 includes three main hubs: machine learning libraries 1306, integration support 1308 and Spark core 1310. These hubs translate each of the three goals of a big-data platform: volume 1312, velocity 1314, and variety 1316.

Volume 1312 represents a huge volume of data received in various forms such as medical notes, and instrument feeds, to name a few, often received in time series or as continuous feed, and other data sources. This received data is stored, normalized, harvested and eventually ingested using framework 1300. These requirements are translated using Integration Support 1308. In this example embodiment, database 202 is primarily implemented using Cassandra and uses the Hadoop File System hosted on an Amazon EC2 Virtual instance. Cassandra allowing queries to be run using SparkSQL and also provides support with standard data transport protocols such as JSON as may be used to transport data in FIG. 1 of Appendix B of U.S. patent application Ser. No. 62/194,904.

Velocity

Healthcare big-data platform 1302 supports real time data, which may be periodic or asynchronous, and functionality for processing these types of data is realized by exploiting the real time processing framework of Apache Spark 1304. For example, real-time feeds from various medical instruments, such as ECG, EEG, Blood Pressure Monitors or Dialysis Machines, shown as transducers 231 of system 100 in FIG. 2.

Variety

Healthcare big-data platform 1302 supports data from disparate sources that is handled by our big data platform. These are processed by translating them through various modules that connects with ‘core’ Spark modules. One such example is patient notes that contain natural language phrases 602 as shown in FIG. 6. These modules include text handler, query processor (e.g., see FIG. 7) and NoSQL database support. Another example is Speech Processing and Analysis as shown in FIG. 5 of Appendix A of U.S. patent application Ser. No. 62/194,904. These are mapped using a Resilient Distributed Data Set framework as supported by Apache Spark 1304.

Biz Data Analytics

Machine Learning Library 1306 provides access to standard machine learning algorithms such as pattern recognition, time series analysis, and semantic analysis. These algorithms may be used to process data from transducers 231 of FIGS. 2 and 3, big data 450 of FIG. 4 of Appendix A of U.S. patent application Ser. No. 62/194,904, and phrase extraction and concept recognition tool 702 of FIG. 7 of Appendix A of U.S. patent application Ser. No. 62/194,904, for example. Framework 1300 thereby implements intelligence of analytic engine 224 of FIGS. 2, 4 and 5, healthcare analytic engine 124 of FIGS. 1, 2, and 3 of Appendix A of U.S. patent application Ser. No. 62/194,904, and analytic engine 124 of FIG. 1 of Appendix B of U.S. patent application Ser. No. 62/194,904. This described functionality is implemented by framework 1300 to overcome one of the biggest challenges 1320, how to process and generate insight from multiple disparate data sources 1322 within Healthcare big data platform 1302.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. In particular, the following embodiments are specifically contemplated, as well as any combinations of such embodiments that are compatible with one another:
(A1) A health information medical collection, processing, and feedback continuum system, including: a knowledgebase; a plurality of transducers for continuously and/or periodically collecting healthcare data from disparate sources for a plurality of patients; and an analytic engine capable of receiving and processing the healthcare data to continuously and/or periodically update the knowledgebase and to determine a patient medical model from the knowledgebase for one of the plurality of patients.
(A2) The system denoted above as (A1), further including an interactive medical intensity status display for interactively displaying, based upon the patient medical model, one or more of a past medical status, a current medical status, and a predicted medical status of the one patient.
(A3) either of the systems denoted above as (A1) and (A2), each of the plurality of transducers comprising at least one sensor selected from the group comprising: a sound sensor, a vibration sensor, an image sensor; an olfactory sensor, a motion sensor, a taste sensor, a temperature sensor, a humidity, hydration sensor, a compliance sensor, a stiffness sensor, and a pressure sensor.
(A4) Any of the systems denoted above as (A1) through (A3), each of the plurality of transducers comprising at least one sensor selected from the group consisting of a microphone and a camera.
(A5) Any of the systems denoted above as (A1) through (A4), each of the plurality of transducers comprising at least one sensor selected from the group comprising a wearable sensor, and an implanted sensor; each of said plurality of transducers providing information at the time of patient encounter.
(A6) Any of the systems denoted above as (A1) through (A5), the healthcare data being one or more of asked data, evoked data, detected data, symptom data, sign data, lab data, imaging data, test data, and sensory data.
(A7) Any of the systems denoted above as (A1) through (A6), wherein at least one of the plurality of transducers is portable.
(A8) Any of the systems denoted above as (A1) through (A7), wherein at least one of the plurality of transducers is adaptable to receive at least one additional type of sensor.
(A9) Any of the systems denoted above as (A1) through (A8), the healthcare data comprising at least one of audio data, video data, olfactory data, taste data, motion and movement data, temperature data, hydration data, material property data, vibration data, and pressure data.
(A10) Any of the systems denoted above as (A1) through (A9), the analytic engine capable of inferring sentiment of the patient from the healthcare data.
(A11) Any of the systems denoted above as (A1) through (A10), wherein the patient medical model predicts the medical status of the one patient based upon healthcare data of other of the plurality of patients having similar medical status to the one patient.
   (B1) A medical feedback continuum method, including receiving, within a healthcare computer and from disparate sources, healthcare data for a plurality of patients; processing the healthcare data to form normalized healthcare data; storing the normalized healthcare data within a knowledgebase; and processing the knowledgebase to determine a patient medical model for one of the plurality of patients based upon healthcare data of other of the plurality of patients having similar medical conditions to the one patient.
   (B2) The method denoted above as (B1), further including generating a medical intensity status based upon the patient medical model; and displaying the medical intensity status to one of a doctor and the one patient during a consultation between the doctor and the one patient.
(B3) Either method denoted above as (B1) and (B2), further including: determining a predicted healthcare outcome from the patient medical model for when the one patient complies with a prescribed intervention; and displaying, within the medical intensity status, the predicted healthcare outcome.
(B4) Any of the methods denoted above as (B1) through (B3), further including: determining a predicted healthcare outcome from the patient medical model for when the one patient does not comply with a prescribed intervention; and displaying, within the medical intensity status, the predicted healthcare outcome.
(B5) Any of the methods denoted above as (B1) through (B4), the medical intensity status comprising patient wellbeing, patient activity, patient morale, and patient social graph.
(B6) Any of the methods denoted above as (B1) through (B5), the medical intensity status including details of a disease diagnosis for the one patient, wherein the medical intensity status educates the one patient on the effects of the disease.
(B7) Any of the methods denoted above as (B1) through (B6), the step of processing the healthcare data including processing healthcare data of a plurality of patients collected from disparate sources to determine the patient medical model of the patient, the method further including: generating a medical intensity status from the patient medical model; displaying the medical intensity status to a doctor during a consultation of the doctor with the patient; collecting healthcare information during the consultation; processing the collected healthcare information to determine an intended intervention prescribed by the doctor for the patient; predicting an outcome of the intervention based upon analytics of the patient medical model; determining whether the predicted outcome of the intervention is favorable for the patient; and if the predicted outcome of the intervention is not favorable: generating an intervention alert; and sending the intervention alert to the doctor during the consultation.
(B8) The methods denoted above as (B7), the intervention alert comprising the predicted outcome.
(B9) Either method denoted above as (B7) and (B8), wherein the location of the consultation is selected from the group including a consulting room, a home of the patient, a hospital, a care facility, a nursing facility, a rehabilitation facility, a convalescent care center, a skilled nursing facility, an assisted living facility, a long-term care facility, a hospice.
(B10) Any of the methods denoted above as (B7) through (B9), the step of predicting comprising invoking an analytic engine to determine the predicted outcome based upon healthcare data of other of the plurality of patients.

What is claimed is:

1. A health information medical collection, processing, and feedback continuum system, comprising:
a knowledgebase;
a plurality of transducers for continuously and/or periodically collecting healthcare data from disparate sources for a plurality of patients;
an analytic engine capable of receiving and processing the healthcare data to continuously and/or periodically update the knowledgebase and to determine a patient medical model from the knowledgebase for one of the plurality of patients; and
an interactive medical intensity status display for interactively displaying, based upon the patient medical model, one or more of a past medical status, a current medical status, and a predicted medical status of the one patient.

2. The system of claim 1, each of the plurality of transducers comprising at least one sensor selected from the group comprising: a sound sensor, a vibration sensor, an image sensor; an olfactory sensor, a motion sensor, a taste sensor, a temperature sensor, a humidity, hydration sensor, a compliance sensor, a stiffness sensor, and a pressure sensor.

3. The system of claim 1, each of the plurality of transducers comprising at least one sensor selected from the group consisting of a microphone and a camera.

4. The system of claim 1, each of the plurality of transducers comprising at least one sensor selected from the group comprising a wearable sensor, and an implanted sensor; each of said plurality of transducers providing information at the time of patient encounter.

5. The system of claim 1, the healthcare data being one or more of asked data, evoked data, detected data, symptom data, sign data, lab data, imaging data, test data, and sensory data.

6. The system of claim 1, wherein at least one of the plurality of transducers is portable.

7. The system of claim 1, wherein at least one of the plurality of transducers is adaptable to receive at least one additional type of sensor.

8. The system of claim 1, the healthcare data comprising at least one of audio data, video data, olfactory data, taste data, motion and movement data, temperature data, hydration data, material property data, vibration data, and pressure data.

9. The system of claim 1, the analytic engine capable of inferring sentiment of the patient from the healthcare data.

10. The system of claim 1, wherein the patient medical model predicts the medical status of the one patient based upon healthcare data of other of the plurality of patients having similar medical status to the one patient.

11. A medical feedback continuum method, comprising:
receiving, within a healthcare computer and from disparate sources, healthcare data for a plurality of patients;
processing the healthcare data to form normalized healthcare data;
storing the normalized healthcare data within a knowledgebase; and
processing the knowledgebase to determine a patient medical model for one of the plurality of patients based upon healthcare data of other of the plurality of patients having similar medical conditions to the one patient.

12. The method of claim 11, further comprising:
generating a medical intensity status based upon the patient medical model; and
displaying the medical intensity status to one of a doctor and the one patient during a consultation between the doctor and the one patient.

13. The method of claim 12, further comprising:
determining a predicted healthcare outcome from the patient medical model for when the one patient complies with a prescribed intervention; and
displaying, within the medical intensity status, the predicted healthcare outcome.

14. The method of claim 12, further comprising:
determining a predicted healthcare outcome from the patient medical model for when the one patient does not comply with a prescribed intervention; and
displaying, within the medical intensity status, the predicted healthcare outcome.

15. The method of claim 12, the medical intensity status comprising patient wellbeing, patient activity, patient morale, and patient social graph.

16. The method of claim 12, the medical intensity status comprising details of a disease diagnosis for the one patient, wherein the medical intensity status educates the one patient on the effects of the disease.

17. The method of claim 11, the step of processing the healthcare data comprising processing healthcare data of a plurality of patients collected from disparate sources to determine the patient medical model of the patient, the method further comprising:
generating a medical intensity status from the patient medical model;
displaying the medical intensity status to a doctor during a consultation of the doctor with the patient;
collecting healthcare information during the consultation;
processing the collected healthcare information to determine an intended intervention prescribed by the doctor for the patient;
predicting an outcome of the intervention based upon analytics of the patient medical model;
determining whether the predicted outcome of the intervention is favorable for the patient; and
if the predicted outcome of the intervention is not favorable:
generating an intervention alert; and
sending the intervention alert to the doctor during the consultation.

18. (canceled)

19. The method of claim 17, wherein the location of the consultation is selected from the group including a consulting room, a home of the patient, a hospital, a care facility, a nursing facility, a rehabilitation facility, a convalescent care center, a skilled nursing facility, an assisted living facility, a long-term care facility, a hospice.

20. The method of claim 17, the step of predicting comprising invoking an analytic engine to determine the predicted outcome based upon healthcare data of other of the plurality of patients.

21. A medical feedback continuum system, comprising:
a plurality of transducers for collecting medical information of a plurality of patients from disparate sources;
a knowledgebase for storing the medical information; and
an analyzer for processing the knowledgebase to determine a medical intensity status display indicative of health of one of the plurality of patients.
